(19) United States
(12) Patent Application Publication
(10) Pub. No.: US 2022/0002293 A1
(43) Pub. Date: Jan. 6, 2022

(54) 8-CYCLOPENTYL-7-OXO-2-(4-PIPERAZIN-1-YL-PHENYLAMINO)-7, 8-DIHYDRO-PYRIDO [2,3-D]PYRIMIDINE-6-CARBONITRILE AND USES THEREOF IN TREATING PROLIFERATIVE DISORDERS

(71) Applicant: ONCONOVA THERAPEUTICS, INC., Newtown, PA (US)

(72) Inventors: Manoj MANIAR, Fremont, CA (US); Chen REN, Newtown, PA (US); Muralidhar MALLIREDDIGARI, Morton, PA (US)

(73) Assignee: ONCONOVA THERAPEUTICS, INC., Newtown, PA (US)

(21) Appl. No.: 17/292,953

(22) PCT Filed: Nov. 12, 2018
(86) PCT No.: PCT/US2018/060564

§ 371 Date: May 11, 2021

(51) Int. Cl. C07D 471/04; A61P 35/00

(52) U.S. Cl. CPC C07D 471/04; A61P 35/00

(57) ABSTRACT

The novel compound 8-Cyclopentyl-7-oxo-2-(4-piperazin-1-yl-phenylamino)-7,8-dihydro-pyrido[2,3-<i]pyrimidine-6-carbonitrile and pharmaceutically acceptable salts thereof are described as well as methods of their use in the treatment of cellular proliferative disorders including cancer.

### FIELD OF THE INVENTION

The invention relates to compounds and pharmaceutically acceptable salts, pharmaceutical compositions, methods for their preparation, and their use for the treatment of cancer and other cellular proliferative disorders.

### BACKGROUND OF THE INVENTION

Cellular proliferative disorders such as cancer are among the most common causes of death in developed countries. For diseases for which treatments exist, such as cancer, despite continuing advances, the existing treatments have undesirable side effects and limited efficacy. Frequently, tumor cells become resistant to existing treatments. Thus, there is a need for new effective drugs for cellular proliferative disorders, including cancer.

Cyclin dependant kinases (Cdk) along with their partner cyclins are a class of holoenzymes that act as regulators of cell proliferation. Selective Cdk4 inhibitors, such as palbociclib, have recognized anti-proliferative activity and palbociclib has been approved for the treatment of some forms of breast cancer. However, patients taking palbociclib have a high incidence of side effects including neutropenia and resistance. ARK5 (AMPK related protein kinase-5) is another kinase associated with cell growth regulation and the overexpression of this kinase has been associated with tumor invasion and metastasis in numerous forms of cancer and particularly in multiple myeloma.

The present invention is predicated, at least in part, by the discovery that 8-Cyclopentyl-7-oxo-2-(4-piperazin-1-yl-phenylamino)-7,8-dihydro-pyrido[2,3-d]pyrimidine-6-carbonitrile (ON 1232580) had surprisingly improved potency as a multi-specific protein kinase inhibitor. In addition, it was also surprising that 8-Cyclopentyl-7-oxo-2-(4-piperazin-1-yl-phenylamino)-7,8-dihydro-pyrido[2,3-d]pyrimidine-6-carbonitrile was a potent cytotoxic agent across a broad range of cancer cell types and specifically against B-cell non-hodgkins lymphoma, prostate cancer, cervical cancer, and chronic myelogenous leukemia (CML).

### SUMMARY OF THE INVENTION

In a first aspect of the invention, there is provided a compound of Formula I:

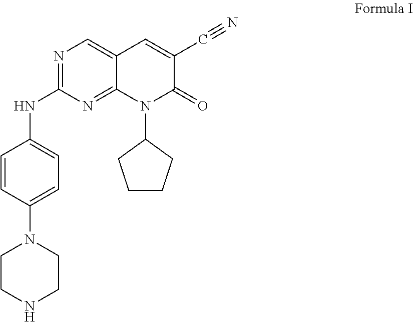 [SMILES: N#Cc1cc2cnc(Nc3ccc(N4CCNCC4)cc3)nc2n(C2CCCC2)c1=O]

and pharmaceutically acceptable salts thereof.

In a second aspect of the invention, there is provided pharmaceutical composition comprising a compound of Formula I, and pharmaceutically acceptable salts thereof, and one or more pharmaceutically acceptable excipients.

In yet another aspect of the invention, there is provided a method of treating proliferative disorders by administering a therapeutically effective amount of compound of Formula I, and pharmaceutically acceptable salts thereof, to a patient in need thereof.

In yet another aspect of the invention, there is provided a method of treating cancer by administering a therapeutically effective amount of compound of Formula I, and pharmaceutically acceptable salts thereof, to a patient in need thereof.

In yet another aspect of the invention, there is provided a pharmaceutical composition for use in treating cancer and proliferative disorders by administering the pharmaceutical composition comprising a therapeutically effective amount of compound of Formula I, and pharmaceutically acceptable salts thereof, to a patient in need thereof.

The present invention also provides a method of treating an individual for a cellular proliferative disorder, comprising administering to the individual an effective amount of at least one compound according to Formula I, or a salt thereof.

In certain embodiments, the cellular proliferative disorder is selected from the group consisting of hemangiomatosis in newborn, secondary progressive multiple sclerosis, atherosclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis, benign proliferative breast disease, benign prostatic hyperplasia, X linked lymphocellular proliferative disorder, post transplantation lymphocellular proliferative disorder, macular degeneration, retinopathies, proliferative vitreoretinopathy and non cancerous lymphocellular proliferative disorders.

In particular embodiments, the cellular proliferative disorder is cancer. In some embodiments, the cancer is selected from the group consisting of ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia; lymphoma, including B-cell lymphoma, non-Hodgkin's lymphoma, and Mantle Cell lymphoma.

The present invention further provides a method of inducing apoptosis of cancer cells in an individual afflicted with cancer, comprising administering to the individual an effective amount of compound of Formula I, or a salt thereof.

In some embodiments, the cancer cells are tumor cells. In particular embodiments, the tumor cells are selected from the group consisting of ovarian, cervical, uterine, vaginal, breast, prostate, testicular, lung, renal, colorectal, stomach, adrenal, mouth, esophageal, hepatic, gall bladder, bone, bone marrow, lymphatic, eye, skin, and brain tumor cells.

The present invention further provides a method of inhibiting kinase activity in a mammal in need of such treatment, said method comprising administering a therapeutically effective amount of a compound of Formula I, or a pharmaceutically acceptable salt thereof. In certain embodiments, one or more of the following kinases are inhibited: ARK5, CDK4/6, ABL1, FGFR1, FLT3, FLT4/VEGFR3, FYN, PDGFRb, and RET. Preferably, two or more, or three or more of said kinases are inhibited.

In yet another aspect of the invention, there is provided the use of 8-Cyclopentyl-7-oxo-2-(4-piperazin-1-yl-phenylamino)-7,8-dihydro-pyrido[2,3-d]pyrimidine-6-carbonitrile, and pharmaceutically acceptable salts thereof, for the manufacture of a medicament for treating cancer and proliferative disorders, wherein the medicament comprises a therapeutically effective dose.

In another aspect, there is provided the compound 8-Cyclopentyl-7-oxo-2-(4-piperazin-1-yl-phenylamino)-7,8-dihydro-pyrido[2,3-d]pyrimidine-6-carbonitrile, and pharmaceutically acceptable salts thereof, for use in the treatment of conditions mediated by abnormal cell proliferation. Preferably, such condition is cancer; or such condition is cancer is selected from ovarian, cervical, uterine, vaginal, breast, prostate, testicular, lung, renal, colorectal, stomach, adrenal, mouth, esophageal, hepatic, gall bladder, bone, bone marrow, leukemia, lymphatic, eye, skin, and brain cancer; or such condition is cancer selected from leukemia, including acute myeloid leukemia, chronic myeloid leukemia, acute lymphoid leukemia, and chronic lymphoid leukemia; or such condition is cancer selected from lymphoma, including B-cell lymphoma, non-Hodgkin's lymphoma, and Mantle Cell lymphoma; or such condition is cancer selected from breast, multiple myeloma, B-Cell lymphoma, prostate, and cervical cancer; or such condition is hormone receptor positive, human epidermal growth factor receptor 2 negative advanced or metastatic breast cancer. The medical treatment includes treating human with a therapeutically effective amount of the compounds of Formula I and its pharmaceutically acceptable salts.

All publications and patent applications mentioned in this specification are incorporated by reference in their entirety to the same extent as if each individual publication or patent application was specifically and individually indicated to be incorporated by reference.

### DETAILED DESCRIPTION OF THE INVENTION

The present invention provides the novel compound 8-Cyclopentyl-7-oxo-2-(4-piperazin-1-yl-phenylamino)-7,8-dihydro-pyrido[2,3-d]pyrimidine-6-carbonitrile (ON 1232580), and pharmaceutically acceptable salts thereof. The inventors found that 8-Cyclopentyl-7-oxo-2-(4-piperazin-1-yl-phenylamino)-7,8-dihydro-pyrido[2,3-d]pyrimidine-6-carbonitrile had surprisingly improved potency as a multi-specific protein kinase inhibitor. In addition, it was also surprising that 8-Cyclopentyl-7-oxo-2-(4-piperazin-1-yl-phenylamino)-7,8-dihydro-pyrido[2,3-d]pyrimidine-6-carbonitrile was a potent cytotoxic agent across a broad range of cancer cell types and specifically against B-cell non-Hodgkin's lymphoma, prostate cancer, cervical cancer, and chronic myelogenous leukemia.

The compounds and compositions of the invention are believed to selectively inhibit proliferation of cancer cells, and kill various tumor cell types. The compounds of the invention inhibit various protein kinases. The compounds of the present invention show surprising potency inhibiting ARK5, CDK4/6, as well as ABL1, CDK9, FGFR1, FLT3, VEGFR3, FYN, PDGFRb, and RET as described in further detail below.

The compounds of the invention are believed to inhibit the proliferation of tumor cells and induce cell death. Cell death results from the induction of apoptosis. The compounds are believed effective against a broad range of tumor types, including but not limited to the following: ovarian cancer, breast cancer, prostate cancer, lung cancer, renal cancer, colorectal cancer, brain cancer, lymphoma and leukemia. Surprisingly, ON 1232580 was a potent killer of GRANTA-519 cells which are a B-Cell non-Hodgkin's lymphoma cell line, specifically a Mantle cell lymphoma. In contrast, cisplatin was not very toxic to GRANTA-519 cells. As shown by cytotoxicity studies, ON 1232580 is also a potent inhibitor of DU 145 cells (prostate cancer), HeLa cells (cervical cancer), and K-562 cells (human myelogenous leukemia).

The compounds are also believed useful in the treatment of non-cancer cellular proliferative disorders, including but not limited to the following: hemangiomatosis in newborn, secondary progressive multiple sclerosis, atherosclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis, benign proliferative breast disease, benign prostatic hyperplasia, X linked lymphocellular proliferative disorder, post transplantation lymphocellular proliferative disorder, macular degeneration, retinopathies, proliferative vitreoretinopathy and non-cancerous lymphocellular proliferative disorders

### Terms

Unless defined otherwise, all technical and scientific terms used herein have the same meaning as is commonly understood by one of skill in the art to which the invention(s) belong.

The terms used in the specification have the following meanings:

As used herein, the term “pharmaceutical composition” means, for example a mixture containing a specified amount of a therapeutic compound, e.g. a therapeutically effective amount, in a pharmaceutically acceptable carrier to be administered to a mammal, e.g. a human in order to treat a disease.

As used herein, the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problematic complications commensurate with a reasonable benefit/risk ratio.

As used in the specification and the appended claims, the singular forms “a,” “an” and “the” include plural referents unless the context clearly dictates otherwise.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms. The terms are understood to mean the management and care of a patient for the purpose of combating a disease, condition, or disorder.

As used herein, “individual” (as in the subject of the treatment) means both mammals and non-mammals. Mammals include, for example, humans; non-human primates, e.g. apes and monkeys; cattle; horses; sheep; and goats. Non-mammals include, for example, fish and birds.

The expression “effective amount”, when used to describe therapy to an individual suffering from a cancer or other cellular proliferative disorder, refers to the amount of a compound according to Formula I that inhibits the abnormal growth or proliferation, or alternatively induces apoptosis of cancer cells, preferably tumor cells, resulting in a therapeutically useful and/or selective cytotoxic effect on proliferative cells.

The term “cellular proliferative disorder” means a disorder wherein unwanted cell proliferation of one or more subsets of cells in a multicellular organism occurs. In some such disorders, cells are made by the organism at an atypically accelerated rate.

Compounds of the Invention

Compounds of the invention include the compounds of Formula I:

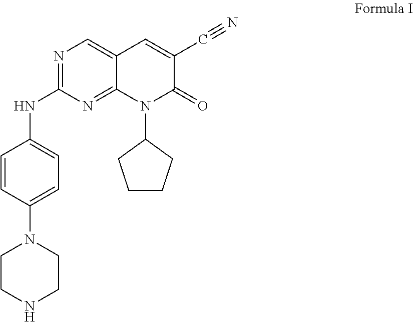 [SMILES: N#Cc1cc2cnc(Nc3ccc(N4CCNCC4)cc3)nc2n(C2CCCC2)c1=O]

and pharmaceutically acceptable salts thereof.

In one aspect, preferred is compounds of Formula I and pharmaceutically acceptable salts selected from salts of inorganic acids, for example, HCl, and organic acids, for example, lactic acid.

In other preferred embodiments, the compound of Formula I, or any of the embodiments thereof, is a pure compound prepared by chemical synthesis and confirmed for purity by analytical methodology. In other preferred embodiments, the compound of Formula I, and compositions containing the compounds, including pharmaceutical compositions, are substantially free of pharmaceutically unacceptable contaminants. A pharmaceutically unacceptable contaminant is a substance which, if present in more than an insubstantial amount, would render the compound or composition unsuitable for use as a pharmaceutical for therapeutic administration. Examples include toxic materials such as halogenated solvents and heavy metals, and potentially infectious materials such as bacteria, fungi, viruses, and bacterial and fungal spores.

Salts of Compounds of Formula I

The compounds of the present invention may take the form of salts. The term “salts” embraces addition salts of compound of Formula I which are compounds of the invention. The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic carboxylic and sulfonic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoroacetic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

In one aspect, preferred salts of the compound of Formula I include hydrochloride and lactate. Lactate salts include salts prepared with (R)-, (S)- and (RS)-Lactic acid isomers.

All of these salts may be prepared by conventional means from the corresponding compound according to Formula I and the appropriate acid. Preferably the salts are in crystalline form, and preferably prepared by crystallization of the salt from a suitable solvent. The person skilled in the art will know how to prepare and select suitable salt forms for example, as described in Handbook of Pharmaceutical Salts: Properties, Selection, and Use By P. H. Stahl and C. G. Wermuth (Wiley-VCH 2002).

Methods of Making the Compounds of the Invention

Example 1 below provides a method for the synthesis of compounds according to Formula I and its salts. The compounds, including salts of the invention and intermediates may be isolated from their reaction mixtures and purified by standard techniques such as filtration, liquid-liquid extraction, solid phase extraction, distillation, recrystallization or chromatography, including flash column chromatography, or HPLC. The preferred method for purification of the compounds according to Formula I or salts thereof comprises crystallizing the compound or salt from a solvent to form, preferably, a crystalline form of the compounds or salts thereof. Following crystallization, the crystallization solvent is removed by a process other than evaporation, for example filtration or decanting, and the crystals are then preferably washed using pure solvent (or a mixture of pure solvents). Preferred solvents for crystallization include water, alcohols, particularly alcohols containing up to four carbon atoms such as methanol, ethanol, isopropanol, and butan-1-ol, butan-2-ol, and 2-methyl-2-propanol, ethers, for example diethyl ether, diisopropyl ether, t-butyl methyl ether, 1,2-dimethoxyethane, tetrahydrofuran and 1,4-dioxane, carboxylic acids, for example formic acid and acetic acid, and hydrocarbon solvents, for example pentane, hexane, toluene, and mixtures thereof, particularly aqueous mixtures such as aqueous ethanol. Other possible solvents include ethyl acetate. Pure solvents, preferably at least analytical grade, and more preferably pharmaceutical grade are preferably used. In a preferred embodiment of the processes of the invention, the products are so isolated. In the compounds of the invention according to Formula I or salt thereof, and pharmaceutical compositions thereof, the compound according to Formula I or salt thereof is preferably in or prepared from a crystalline form, preferably prepared according to such a process.

It will be appreciated by one skilled in the art that the processes described are not the exclusive means by which compounds of the invention may be synthesized and that an extremely broad repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), Comprehensive Heterocyclic Chemistry II, Ed. A. R. Katritzky, C. W. Rees, and E. F. V. Scriven (Pergamon Press, 1996), and Advanced Organic Chemistry, 4^{th} Ed., J. March (John Wiley & Sons, 1992).

Dosage Forms and Administration Routes

8-Cyclopentyl-7-oxo-2-(4-piperazin-1-yl-phenylamino)-7,8-dihydropyrido[2,3-d]pyrimidine-6-carbonitrile, and pharmaceutically acceptable salts thereof can be administered in a wide variety of dosage forms. The compounds of the invention may be administered in the form of a pharmaceutical composition, in combination with a pharmaceutically acceptable carrier. The active ingredient in such formulations may comprise from 0.1 to 99.99 weight percent. “Pharmaceutically acceptable carrier” means any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18^{th} Edition (1990), Mack Publishing Co., Easton, Pa. Suitable dosage forms may comprise, for example, tablets, capsules, solutions, parenteral solutions, troches, suppositories, or suspensions.

For parenteral administration, including injectable administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, its salts and esters, cysteine and its derivatives, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The specific dose of a compound according to the invention to obtain therapeutic benefit for treatment of a cellular proliferative disorder will, of course, be determined by the particular circumstances of the individual patient including the size, weight, age and sex of the patient, the nature and stage of the cellular proliferative disorder, the aggressiveness of the cellular proliferative disorder, and the route of administration of the compound.

For example, a daily dosage from about 0.05 to about 50 mg/kg/day may be utilized, more preferably from about 0.1 to about 10 mg/kg/day. Higher or lower doses are also contemplated as it may be necessary to use dosages outside these ranges in some cases. The daily dosage may be divided, such as being divided equally into two to four times per day daily dosing. The compositions are preferably formulated in a unit dosage form, each dosage containing from about 1 to about 500 mg, more typically, about 10 to about 100 mg of active agent per unit dosage. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The compounds may be administered by any route, including but not limited to oral, rectal, sublingual, buccal, ocular, pulmonary, and parenteral administration, or as an oral or nasal spray (e.g. inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical or subcutaneous administration. The pharmaceutical compositions can be in the form of sterile injectable solutions for parenteral use including concentrated solutions that are diluted prior to administration. Also contemplated within the scope of the invention is the instillation of a drug in the body of the patient in a controlled formulation, with systemic or local release of the drug to occur at a later time. For example, the drug may be localized in a depot for controlled release to the circulation, or for release to a local site of tumor growth.

One or more compounds useful in the practice of the present inventions may be administered simultaneously, by the same or different routes, or at different times during treatment. The compounds may be administered before, along with, or after other medications, including other antiproliferative compounds.

The treatment may be carried out for as long a period as necessary, either in a single, uninterrupted session, or in discrete sessions. The treating physician will know how to increase, decrease, or interrupt treatment based on patient response. According to one embodiment, treatment is carried out for from about four to about sixteen weeks. The treatment schedule may be repeated as required.

The active agent may be formulated for parenteral administration (e.g., by injection, for example via continuous infusion after dilution of the concentrated formulation) and may be presented in unit dose form in ampoules, vials, pre-filled syringes, small volume infusion or in multi-dose containers with or without an added preservative. Parenteral formulations may include suitable anti-oxidants, osmolality adjusting agents, stabilizing agents, and other pharmaceutically acceptable excipients. Suitable anti-oxidants include sodium bisulfite, sodium sulfite, sodium ascorbate, L-cysteine and its derivatives, and sodium thiosulfate, sodium formaldehyde sulfoxylate, citric acid, d,l-α-tocopherol, butylated hydroxy anisole, butylated hydroxy toluene, monothioglycerol, ascorbic acid its salts and esters, and propyl gallate.

The present invention includes a pharmaceutical pack or kit comprising oral dosage forms, vials or ampoules comprising 8-Cyclopentyl-7-oxo-2-(4-piperazin-1-yl-phenylamino)-7,8-dihydro-pyrido[2,3-d]pyrimidine-6-carbonitrile, and pharmaceutically acceptable salts thereof and optionally a container of a suitable liquid as carrier, such as water. Optionally, the pharmaceutical pack or kit can include or have associated with it a notice in the form prescribed by a governmental agency regulating the manufacture, use or sale of pharmaceuticals or biological products, which notice reflects approval by the agency of manufacture, use or sale for human administration.

Methods of Treatment

According to another embodiment of the invention, a method of treating an individual suffering from a cellular proliferative disorder, particularly cancer, is provided, comprising administering to said individual an effective amount of at least one compound according to Formula I, or a pharmaceutically acceptable salt thereof, either alone, or in combination with a pharmaceutically acceptable carrier.

According to another embodiment of the invention, a method of inducing apoptosis of cancer cells, preferably tumor cells, in an individual afflicted with cancer is provided, comprising administering to said individual an effective amount of at least one compound according to Formula I, or a pharmaceutically acceptable salt thereof, either alone, or in combination with a pharmaceutically acceptable carrier.

The invention is also directed to the use in medicine of a compound of Formula I, or a pharmaceutically acceptable salt thereof.

The invention is also directed to compounds of Formula I, and pharmaceutically acceptable salts thereof, for treating a proliferative disorder, or for inducing apoptosis of tumor cells.

The invention is also directed to a medicament comprising a compound of Formula I, or a pharmaceutically acceptable salt thereof, for use in treating a proliferative disorder, or for inducing apoptosis of tumor cells.

The invention is also directed to the use of a compound of Formula I, or a pharmaceutically acceptable salt thereof in the preparation of a medicament for treatment of a cellular proliferative disorder, particularly cancer, or for inducing apoptosis of tumor cells in an individual affected with cancer.

The compounds according to the invention may be administered to individuals (mammals, including animals and humans) afflicted with a cellular proliferative disorder such as cancer, malignant and benign tumors, blood vessel proliferative disorders, autoimmune disorders, and fibrotic disorders. In a particular embodiment of the invention, the individual, subject, or patient treated is a human.

The compounds are believed effective against a broad range of tumor types, including but not limited to the following: ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia; lymphoma, including B cell lymphoma, non-Hodgkin's lymphoma, and Mantle Cell lymphomas.

More particularly, cancers that may be treated by the compounds, compositions and methods of the invention include, but are not limited to, the following:

Breast cancers, including, for example, ductal carcinoma, tubular carcinoma, medullary carcinoma, mucinous carcinoma, papillary carcinoma, and cribriform carcinoma;

cardiac cancers, including, for example sarcoma, e.g., angiosarcoma, fibrosarcoma, rhabdomyosarcoma, and liposarcoma; myxoma; rhabdomyoma; fibroma; lipoma and teratoma;

lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma;

gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma;

genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma;

liver cancers, including, for example, hepatoma, e.g., hepatocellular carcinoma; cholangiocarcinoma; hepatoblastoma; angiosarcoma; hepatocellular adenoma; and hemangioma;

bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors;

nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma;

gynecological cancers, including, for example, cancers of the uterus, e.g., endometrial carcinoma; cancers of the cervix, e.g., cervical carcinoma, and pre-tumor cervical dysplasia; cancers of the ovaries, e.g., ovarian carcinoma, including serous cystadenocarcinoma, mucinous cystadenocarcinoma, unclassified carcinoma, cone-thecal cell tumors, Sertoli-Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma;

hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenström's macroglobulinemia;

skin cancers, including, for example, malignant melanoma, basal cell carcinoma, squamous cell carcinoma, Kaposi' s sarcoma, moles dysplastic nevi, lipoma, angioma, dermatofibroma, keloids, psoriasis; and

adrenal gland cancers, including, for example, neuroblastoma.

Cancers may be solid tumors that may or may not be metastatic. Cancers may also occur, as in leukemia, as a diffuse tissue. Thus, the term “tumor cell”, as provided herein, includes a cell afflicted by any one of the above identified disorders.

The compounds are also believed useful in the treatment of non-cancer cellular proliferative disorders, that is, cellular proliferative disorders which are characterized by benign indications. Such disorders may also be known as “cytoproliferative” or “hyperproliferative” in that cells are made by the body at an atypically elevated rate. Non-cancer cellular proliferative disorders believed treatable by compounds according to the invention include, for example: hemangiomatosis in newborn, secondary progressive multiple sclerosis, atherosclerosis, chronic progressive myelodegenerative disease, neurofibromatosis, ganglioneuromatosis, keloid formation, Paget's disease of the bone, fibrocystic disease of the breast, uterine fibroids, Peyronie' s disease, Dupuytren's disease, restenosis, benign proliferative breast disease, benign prostatic hyperplasia, X-linked lymphocellular proliferative disorder (Duncan disease), post-transplantation lymphocellular proliferative disorder (PTLD), macular degeneration, and retinopathies, such as diabetic retinopathies and proliferative vitreoretinopathy (PVR)

Other non-cancer cellular proliferative disorders believed treatable by compounds according to the invention include the presence of pre-cancerous lymphoproliferative cells associated with an elevated risk of progression to a cancerous disorder. Many non-cancerous lymphocellular proliferative disorders are associated with latent viral infections such as Epstein-Barr virus (EBV) and Hepatitis C. These disorders often begin as a benign pathology and progress into lymphoid neoplasia as a function of time.

Acquired Resistance Cancers and Combination Therapy

According to one embodiment of the invention, the compound of Formula I, and pharmaceutically acceptable salts thereof can be administered to drug resistant cancer patients, who have tumor cells that are resistant to one or more specific anticancer agents.

According to another embodiment of the invention, the compound of Formula I, and pharmaceutically acceptable salts thereof can be administered to a cancer patient prior to, concomitant with, and or subsequent to the administration of another cancer agent.

Examples of anticancer agents to which resistance can develop and/or which can be used in combination with the Compound of Formula I and pharmaceutically acceptable salts thereof include, cytotoxic agents, chemotherapeutic agents (including alkylating agents, antimetabolites, anthracyclines, alkaloids, topoisomerase inhibitors, monoclonal antibodies, among others), CDK4/6 inhibitors including palbociclib, erythropoiesis modulating agents comprising ESAs including EPO (endogenous, recombinant and/or synthetic EPO), epoetin alfa, Procrit, Epogen, epoetin beta, darbepoetin alfa, and/or methoxy polyethylene glycol-epoetin beta; DNA methyltransferase inhibitors (including azacitidine, decitabine, 5-fluoro-2′-deoxycitidine, 5,6-dihydro-5-azacytidine, zebularine, fazarabine, hydralizine, procaine, procainamide, epigallocatechin gallate, psammaplin A, or (S)-2-(1,3-dioxo-1,3-dihydro-isoindol-2-yl)-3-(1H-indol-3-yl)-propionic acid, or a pharmaceutically acceptable salt thereof), immunomodulators such as lenalidomide, among others.

Failure of a patient's cancer to respond to a specific therapy can result from one of two general causes: host factors and specific genetic or epigenetic alterations in the cancer cells. Host factors include poor absorption or rapid metabolism or excretion of a drug, resulting in low serum levels; poor tolerance to effects of a drug, especially in elderly patients, resulting in a need to reduce doses below optimal levels; inability to deliver a drug to the site of a tumor, as could occur with bulky tumors or with biological agents of high molecular weight and low tissue penetration such as monoclonal antibodies and immunotoxins. In addition, various alterations in the host-tumor environment can affect the response of the tumor to cancer therapy, these effects include local metabolism of a drug by normal cells, abnormal and/or unusual features of the tumor blood supply that may affect transit time of drugs within tumors, among others.

The pharmaceutical composition of the invention and the therapeutic regimen as claimed are effective to overcome one or more of the aforementioned obstacles in cancer therapy by raising the tolerance or resistance to anticancer agents or therapies. Accordingly, the method of the invention provides additional tools for effective cancer therapy to combat and defeat drug resistance in cancer patients.

Multidrug resistance, the principal mechanism by which many cancers develop resistance to chemotherapy drugs, is a major factor in the failure of many forms of chemotherapy. It affects patients with a variety of blood cancers and solid tumors. Tumors usually consist of mixed populations of malignant cells, some of which are drug-sensitive while others are drug-resistant. Chemotherapy can destroy drug-sensitive cells, but leaves behind a higher proportion of drug-resistant cells. As the tumor begins to grow again, chemotherapy may fail because the remaining tumor cells are now drug resistant.

Resistance to therapy has been correlated to the presence of at least two molecular “pumps” in tumor-cell membranes that actively expel chemotherapy drugs from the interior. This allows tumor cells to avoid the toxic effects of the drug or molecular processes within the nucleus or the cytoplasm. The two pumps commonly found to confer chemo-resistance in cancer are P-glycoprotein and the so-called multidrug resistance-associated protein (MRP). Because of their function and importance, they are the targets of several anticancer efforts.

According to one embodiment, the compositions and methods of the invention overcome resistance to DNA methyltransferase inhibitors, ESAs, or a combination thereof.

The methods and compositions of the invention are useful in treatment of cancer and the anemia associated with cancer, specifically in patients who have acquired resistance to exogenous erythropoietin (EPO). Resistance to exogenous EPO is associated with an increased risk of death. Anemia in cancer patients rises through many different mechanism of actions and pathways, it can be the direct effects of the cancer cells in the body, as a result of biologically active products of the cancer cells, or as a consequence of the treatment of cancer. There is also an association between anemia and progression of blood cancers. The main causes of anemia are deficient production of erythropoietin (EPO), iron deficiency, and a chronic disease with endogenous EPO resistance. Up to 10% of patients receiving EPO are hyporesponsive to therapy and require large doses of the agent. Proinflammatory cytokines antagonize the action of EPO by exerting an inhibitory effect on erythroid progenitor cells and by disrupting iron metabolism. See, U.S. Pat. No. 8,664,272 B2.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Synthesis of 8-Cyclopentyl-7-oxo-2-(4-piperazin-1-yl-phenylamino)-7,8-dihydropyrido[2,3-d]pyrimidine-6-carbonitrile (ON 1232580)

Synthetic Scheme for the Compound of Formula I:

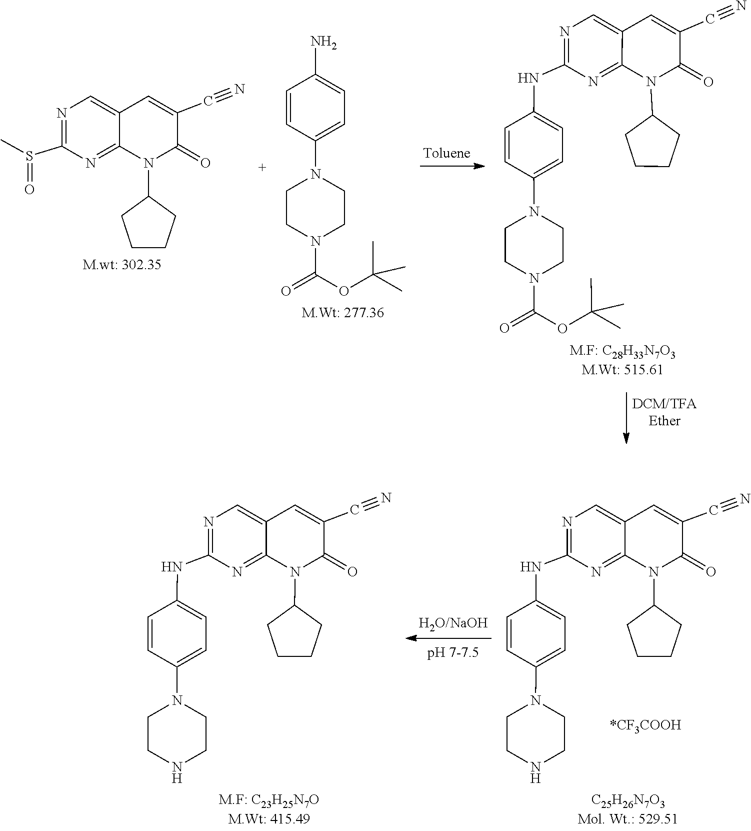 [SMILES: CS(=O)c1ncc2cc(C#N)c(=O)n(C3CCCC3)c2n1 ; CC(C)(C)OC(=O)N1CCN(c2ccc(N)cc2)CC1 ; CC(C)(C)OC(=O)N1CCN(c2ccc(Nc3ncc4cc(C#N)c(=O)n(C5CCCC5)c4n3)cc2)CC1 ; N#Cc1cc2cnc(Nc3ccc(N4CCNCC4)cc3)nc2n(C2CCCC2)c1=O ; N#Cc1cc2cnc(Nc3ccc(N4CCNCC4)cc3)nc2n(C2CCCC2)c1=O]

### Synthesis of 4-[4-(6-Cyano-8-cyclopentyl-7-oxo-7,8-dihydro-pyrido[2,3-d]pyrimidin-2-ylamino)-phenyl]-piperazine-1-carboxylic acid tert-butyl ester (ON 1232570)

4-(4-Aminophenyl)-Piperazine-1-carboxylic acid tert-butylester (1.2 g, 3.96 mmol) was dissolved in toluene (25 mL) and stirred for 10 min. 8-Cyclopentyl-2-methanesulfinyl-7-oxo-7,8-dihydro-pyrido[2,3-d]pyrimidine-6-carbonitrile (1.0 g, 3.31 mmol) was added to the above solution, washed the flask with 10 mL toluene and heated to 70-80° C. and maintained for 4 h. After completion of the reaction monitored by mass spec, the reaction mixture cooled to room temperature and left overnight. The solid formed was filtered, washed with toluene (20 mL), and dried under vacuum. Yield: 1.56 g (91%).

¹H NMR (300 Hz, CDCl₃): δ 1.51 (s, 9H, 3×CH₃), 1.64 (br s, 4H, 2×CH₂), 1.88 (br s, 2H, CH₂), 2.30 (br s, 2H, CH₂), 3.01 (t, 4H, J=10.2, 7.5 Hz, 2×CH₂), 3.63 (t, 4H, J=5.4, 4.8 Hz, 2×CH₂), 5.86 (t, 1H, J=8.4, 8.1 Hz, CH), 6.98 (d, 2H, J=9.0 Hz, Ar—H), 7.47 (d, 2H, J=9.0 Hz, Ar—H), 7.98 (s, 1H, Ar—H) and 8.58 (s, 1H, Ar—H.

Mass: m/z 516.10

### Synthesis of 8-Cyclopentyl-7-oxo-2-(4-piperazin-1-yl-phenylamino)-7,8-dihydro-pyrido[2,3-d]pyrimidine-6-carbonitrile trifluoroacetate (ON 1232580)

4-[4-(6-Cyano-8-cyclopentyl-7-oxo-7,8-dihydro-pyrido[2,3-d]pyrimidin-2-yl-amino)-phenyl]-piperazine-1-carboxylic acid tert-butyl ester (0.75 g, 14.5 mmol) was dissolved in dichloromethane (90 mL) and cooled to 0° C. To the cooled solution under stirring 19.41 g (170.23 mmol) of trifluoroacetic acid dissolved in 30 mL dichloromethane) was added slowly to the cooled solution over a period of 30 min and maintained for 4 h at 0° C. After completion of the reaction monitored by mass spec, the solvent was evaporated under vacuum at room temperature and the residue is diluted with diethyl ether (100 mL) and stirred for 15 min at 0° C. The solid formed was filtered, washed with diethyl ether (50 mL) and dried under vacuum and used as is in the next step. Crude yield: 0.85 g (Quantitative).

¹H NMR (300 Hz, D₂O): δ 1.38 (br s, 4H, 2×CH₂), 1.68 (br s, 4H, 2×CH₂), 3.12 (br s, 4H, 2×CH₂), 3.28 (br s, 4H, 2×CH2), 5.03 (br s, 1H, CH), 6.37 (br s, 2H, Ar—H), 6.66 (d, 2H, J=7.5 Hz, Ar—H), 7.71 (s, 1H, Ar—H) and 8.12 (s, 1H, Ar—H).

Mass: m/z 416.10 (mass spec shows as free base)

### Synthesis of Compound of Formula I as free base, 8-Cyclopentyl-7-oxo-2-(4-piperazin-1-yl-phenylamino)-7,8-dihydro-pyrido[2,3-d]pyrimidine-6-carbonitrile (ON 1232580 free base)

8-Cyclopentyl-7-oxo-2-(4-piperazin-1-yl-phenylamino)-7,8-dihydro-pyrido[2,3-d]pyrimidine-6-carbonitrile trifluoroacetic acid (0.045 g) was dissolved in water (15 mL) and stirred for 10 min. The dissolved solution was filtered and cooled to 0° C. 7% sodium hydroxide solution was added (6-7 drops) until the pH of the solution was adjusted between 7.0-7.5 and the solution maintained for 1 h at 0° C. The solid formed was filtered, washed with water (5 mL), and dried under vacuum and left overnight. Yield: 0.020 g (57.1%).

¹H NMR (300 Hz, DMSO-d₆): δ 1.57 (br s, 2H, CH₂), 1.79 (br s, 4H, 2×CH₂), 2.20 (br s, 2H, CH₂), 2.84 (br s, 4H, 2×CH₂), 3.02 (br s, 4H, 2×CH₂), 5.73 (br s, 1H, CH), 6.94 (d, 2H, J=8.1 Hz, Ar—H), 7.49 (d, 2H, J=8.7 Hz, Ar—H), 8.53 (s, 1H, Ar—H), 8.78 (s, 1H, Ar—H) and 10.38 (br s, 1H, NH).

Mass: m/z 416.10

### Example 2

### Kinase Inhibition of 8-Cyclopentyl-7-oxo-2-(4-piperazin-1-yl-phenylamino)-7,8-dihydropyrido[2,3-d]pyrimidine-6-carbonitrile (ON 123580)

Palbociclib has the structure:

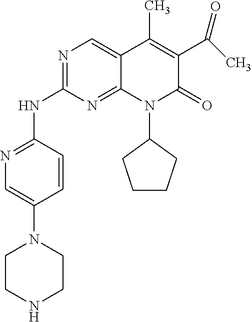 [SMILES: CC(=O)c1c(C)c2cnc(Nc3ccc(N4CCNCC4)cn3)nc2n(C2CCCC2)c1=O]

According to the National Cancer Institute, palbociclib is an orally available pyridopyrimidine-derived cyclin-dependent kinase (CDK) inhibitor with potential antineoplastic activity. In particular, palbociclib selectively inhibits cyclin-dependent kinases (particularly Cdk4/cyclin D1 kinase), which may inhibit retinoblastoma (Rb) protein phosphorylation, which prevents Rb-positive tumor cells from entering the S phase of the cell cycle (arrest in the G1 phase). This results in suppression of DNA replication and decreased tumor cell proliferation.

The compounds in Table 1 were tested for the ability to inhibit the kinase activity of the listed protein kinases. Compounds were tested in 6 dose IC₅₀ mode with 10-fold serial dilution starting at 100 μM. As a Control, Staurosporine, a known protein kinase inhibitor, was tested in 10 dose IC₅₀ mode with 4-fold serial dilution starting at 20 μM. Reactions were carried out in 10 μM ATP. An IC₅₀ value less than 1 nM or higher than 100 μM is estimated based on the best curve fitting available.

TABLE 1
Compound IC50 (M)

| Kinase | ON 1232580 | Palbociclib | Stauro-sporine |
|---|---|---|---|
| ABL1 | 2.01E−08 | 2.02E−05 | 5.53E−08 |
| ARK5/NUAK1 | 2.28E−09 | 1.91E−06 | 5.24E−10 |
| CDK4/cyclin D1 | <1.00E−09 | 1.06E−09 | 9.25E−09 |
| CDK4/cyclin D3 | 2.77E−09 | 3.67E−09 | 2.30E−08 |
| CDK6/cyclin D1 | <1.00E−09 | 5.00E−10 | 3.87E−09 |
| CDK6/cyclin D3 | 2.97E−09 | 3.87E−09 | 2.92E−08 |
| CDK9/cyclin K | 8.06E−09 | 2.52E−07 | 1.05E−08 |
| FGFR1 | 3.09E−08 | 2.14E−05 | 4.91E−09 |
| FLT3 | 4.25E−09 | 7.79E−07 | 9.11E−10 |
| FLT4/VEGFR3 | 6.97E−09 | 1.72E−05 | 1.63E−09 |
| FYN | 3.29E−08 | 1.89E−05 | 1.32E−09 |
| PDGFRb | 6.12E−09 | 5.33E−05 | 1.41E−09 |
| PKG2/PRKG2 | >1.00E−04 | | 3.03E−09 |
| RET | 8.13E−09 | 7.25E−06 | 2.92E−09 |

Abbreviations: ABL1=Abelson tyrosine protein kinase 1; ARK5=AMPK related protein kinase 5; CDK=Cyclin dependent kinases; FGFR1=fibroblast growth factor receptor 1; FLT3=fms like tyrosine kinase 3; FLT4=fms like tyrosine kinase 4; VEGFR3=vascular endothelial growth factor receptor 3; FYN=fyn tyrosine protein kinase; PDGFRb=platelet derived growth factor receptor beta; PGK2=protein kinase G 2; RET=receptor tyrosine kinase

As observed from Table 1, ON 1232580 has a significantly different protein kinase inhibition profile as compared to palbociclib and is surprisingly more potent as a multi-specific protein kinase inhibitor, against ARK5, CDK9/Cyclin K and CDK6/cyclin D3, for example.

### Example 3

### Cytotoxicity Assay in Cancer Cell Lines Comparing Activity of 8-Cyclopentyl-7-oxo-2-(4-piperazin-1-yl-phenylamino)-7,8-dihydropyrido[2,3-d]pyrimidine-6-carbonitrile (ON 123580) to Cisplatin as Control Cytotoxic Agent

The compounds were tested in four cancer cells lines: DU 145 (human prostate cancer), GRANTA-519 (human B Cell lymphoma), HeLa (human cervical cancer), and K-562 (human chronic myelogenous leukemia). Surprisingly, ON 123580 was a potent cytotoxic agent across all four cell lines including against human B-Cell lymphoma cells.

Reagents and Materials:

Fetal Bovine Serum (FBS), (Cat #FND500, ExCell Bio. Store at −20° C.)

96-Well Flat Clear Bottom Black Polystyrene TC-Treated Microplates (Cat #3340, Corning).

CellTiter-Glo® Luminescent Cell Viability Assay (Cat #G7572, Promega. Store at −20° C.) Substrate is sufficient for 1,000 assays at 100 μL per assay in 96-well plates.

Including:

1×100 mL CellTiter-Glo® Buffer

1×vial CellTiter-Glo® Substrate (lyophilized)

The CellTiter-Glo® Luminescent Cell Viability Assay is a homogeneous method to determine the number of viable cells in culture based on quantitation of the ATP present. which signals the presence of metabolically active cells. The homogeneous assay procedure involves adding a single reagent (CellTiter-Glo® Reagent) directly to cells cultured in serum-supplemented medium. The homogeneous “add-mix-measure” format results in cell lysis and generation of a luminescent signal proportional to the amount of ATP present. The amount of ATP is directly proportional to the number of cells present in culture. The assay generates a luminescent signal, produced by the luciferase reaction.

Reagent Preparation
a. Thaw the CellTiter-Glo Buffer, and equilibrate to room temperature prior to use. For convenience, the CellTiter-Glo Buffer may be thawed and stored at room temperature for up to 48 h prior to use.
b. Equilibrate the lyophilized CellTiter-Glo Substrate to room temperature prior to use.
c. Transfer the appropriate volume (100 mL) of CellTiter-Glo Buffer into the amber bottle containing CellTiter-Glo Substrate to reconstitute the lyophilized enzyme/substrate mixture. This forms the CellTiter-Glo Reagent.
   Note: The entire liquid volume of the CellTiter-Glo Buffer bottle may be added to the CellTiter-Glo Substrate vial.
d. Mix by gently vortexing, swirling or by inverting the contents to obtain a homogeneous solution. The CellTiter-Glo Substrate should go into solution easily in less than one minute.

Determination of the Half Maximal Inhibition Concentration IC50:
1. Harvest cells during the logarithmic growth period and count cell number using Count-star.
2. Adjust cell concentrations to 4.44×10⁴ cells/mL with respective culture medium.
3. Add 90 μL cell suspensions to two 96-well plates (plates A and B) with the final cell density of 4×10³ cells/well. (cell concentration is adjusted according to the data base or density optimization assay.)

Next day: For the Plates of T0 Reading:
1) Add 10 μL culture medium to each well of plate A for T0 reading.
2) Equilibrate the plate and its content at RT for approximately 30 min.
3) Add 50 μL CellTiter-Glo (CTG) reagent to each well.
4) Mix content for 5 min on an orbital shaker to induce cell lysis.
5) Allow the plate to incubate at RT for 20 min to stabilize luminescent signal. Note: Uneven luminescent signal within standard plates can be caused by temperature gradients, uneven seeding of cells or edge effects in multiwell plates.
6) Record luminescence (T0) using EnVision Multi Label Reader.

For the Plates of Test Reading:
1) Prepare 10×solution of test article (Top working concentration: 100 μM of test articles in media with 3.16-fold serial dilutions to achieve 9 dose levels. Starting drug concentration was 40 mM in DMSO with final dilution drug concentration of 4 μM)
2) Prepare 10×reference control solutions Cisplatin (Top working concentration: 100 μM in media with 3.16-fold serial dilutions. (Starting cisplatin (from Hospira Australia) concentration was 3.33 mM) with final dilution drug concentration of 100 nM in cell culture medium).
3) Dispense 10 μL (10×) drug solution of both test article and reference control in each well (triplicate for each drug concentration) of the plate B. (DMSO final concentration in culture medium: 0.25% [v/v]).
4) Incubate the test plate B for 72 h in the humidified incubator at 37° C. with 5% CO₂, and then measured by means of CTG assay.
5) Equilibrate the plate and its content at RT for approximately 30 min.
6) Add 50 μL CellTiter-Glo reagent to each well.
7) Mix contents for 5 min on an orbital shaker to induce cell lysis.
8) Allow the plates to incubate at RT for 20 min to stabilize luminescent signal. Note: Uneven luminescent signal within standard plates can be caused by temperature gradients, uneven seeding of cells or edge effects in multiwall plates.
9) Record luminescence.

In order to calculate absolute IC50 (EC50), a dose-response curve was fitted using nonlinear regression model with a sigmoidal dose response. The formula for calculating surviving rate is shown below and the absolute IC50 (EC50) was calculated according to the dose-response curve generated by GraphPad Prism 5.0.

The surviving rate (%)=(Lum_{Test article}−Lum_{Medium control})/(Lum_{Non-treated}−Lum_{Medium control})×100%.

The IC50 (concentration of drug resulting in 50% of cells killed is summarized in Table 2.

TABLE 2
Summary of Absolute IC50s & Maximal inhibition in 4 cell lines

| Cell | | Absolute IC50(μM) | | % inhibition at top conc. | |
|---|---|---|---|---|---|
| No. | Cell lines | 1232580 | Cisplatin | 1232580 | Cisplatin |
| 1 | DU 145 | 0.93 | 0.96 | 99.94% | 95.44% |
| 2 | GRANTA-519 | 0.40 | 1.68 | 99.97% | 77.25% |
| 3 | HeLa | 1.50 | 0.27 | 99.96% | 99.18% |
| 4 | K-562 | 1.19 | 3.76 | 99.97% | 98.74% |

Surprisingly, ON 1232580 was a potent killer of GRANTA-519 cells which are a B-Cell non-Hodgkin's lymphoma (Mantle Cell) cell line. In contrast, cisplatin was not very toxic to GRANTA-519 cells. As shown by Table 2, ON 1232580 is also a potent inhibitor of DU 145 cells (prostate cancer), HeLa cells (cervical cancer), and K-562 cells (human myelogenous leukemia).

All references cited herein, including patents, patent applications, and publications, are hereby incorporated by reference in their entireties, whether previously specifically incorporated or not.

The various features and embodiments of the present invention, referred to in individual sections above apply, as appropriate, to other sections, mutatis mutandis. Consequently, features specified in one section may be combined with features specified in other sections, as appropriate.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein. Those skilled in the art will recognize, or be able to ascertain using no more than routine experimentation, many equivalents to the embodiments of the invention described herein. Such equivalents are encompassed by the following claims.

What is claimed:

1. A compound according to Formula I, 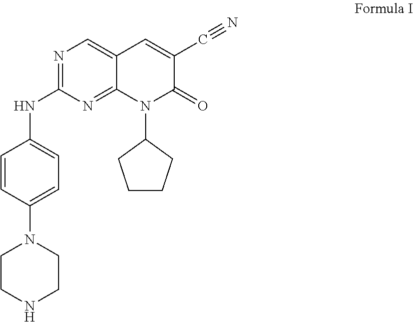 [SMILES: N#Cc1cc2cnc(Nc3ccc(N4CCNCC4)cc3)nc2n(C2CCCC2)c1=O]
and pharmaceutically acceptable salts thereof.

2. A pharmaceutical composition comprising a compound of Formula I 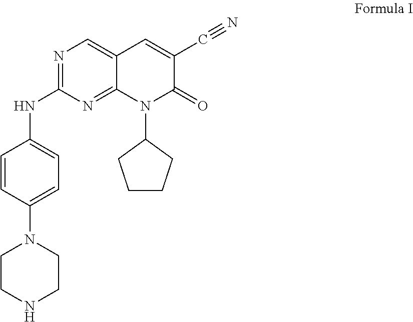 [SMILES: N#Cc1cc2cnc(Nc3ccc(N4CCNCC4)cc3)nc2n(C2CCCC2)c1=O]
and pharmaceutically acceptable salts thereof, and one or more pharmaceutically acceptable excipients.

3. A method of treating conditions mediated by abnormal cell proliferation comprising administering an effective amount of a compound of claim 1 and pharmaceutically acceptable salts thereof to a subject in need thereof.

4. The method of claim 3 wherein said condition is cancer and said subject is human.

5. The method of claim 4 wherein said cancer is selected from ovarian, cervical, uterine, vaginal, breast, prostate, testicular, lung, renal, colorectal, stomach, adrenal, mouth, esophageal, hepatic, gall bladder, bone, leukemia, lymphatic, eye, skin, and brain.

6. The method of claim 5 wherein the cancer is selected from acute myeloid leukemia, chronic myeloid leukemia, acute lymphoid leukemia, and chronic lymphoid leukemia.

7. The method of claim 3 wherein said cancer is lymphoma.

8. The method of claim 7 wherein said lymphoma is non-Hodgkin's B-Cell lymphoma.

9. The method of claim 8 wherein said lymphoma is Mantle Cell lymphoma.

10. The method according to claim 3, wherein said subject is human and the cellular proliferative disorder is selected from the group consisting of hemangiomatosis in newborn, secondary progressive multiple sclerosis, atherosclerosis, chronic progressive myelodegenerative disease, neurofibromatosis, ganglioneuromatosis, keloid formation, Paget' s disease of the bone, fibrocystic disease of the breast, uterine fibroids, Peyronie's disease, Dupuytren's disease, restenosis, benign proliferative breast disease, benign prostatic hyperplasia, X linked lymphocellular proliferative disorder, post transplantation lymphocellular proliferative disorder, macular degeneration, retinopathies, proliferative vitreoretinopathy and non-cancerous lymphocellular proliferative disorders.

11. A method of inhibiting kinase activity in a mammal, comprising administering a therapeutically effective amount of a compound of claim 1, or a pharmaceutically acceptable salt thereof, to a patient in need of such treatment.

12. The method of claim 11 wherein said patient is human suffering from breast cancer, multiple myeloma, B-cell lymphoma, prostate cancer, or cervical cancer.

13. A method of inducing apoptosis of cancer cells in an individual afflicted with cancer, comprising administering to the individual an effective amount of compound of claim 1, or a salt thereof.

14. The method of claim 13 wherein said individual is human afflicted with cancer selected from breast cancer, multiple myeloma, B-cell lymphoma, prostate cancer, and cervical cancer.

15. The method of claim 5 wherein said cancer is selected from breast cancer, multiple myeloma, B-cell lymphoma, prostate cancer, and cervical cancer.

16. The method of claim 5, wherein said cancer is breast cancer.

17. The method of claim 16 wherein the cancer is hormone receptor-positive, human epidermal growth factor receptor 2-negative advanced or metastatic breast cancer.
